(19) United States
(12) Patent Application Publication
(10) Pub. No.: US 2005/0251143 A1
(43) Pub. Date: Nov. 10, 2005

(54) Surgical system and method of use for soft tissue fixation to bone

(76) Inventor: David G. Dillard, Hartwell, GA (US)

Correspondence Address: JOHN S. PRATT, ESQ;KILPATRICK STOCKTON, LLP, 1100 PEACHTREE STREET, ATLANTA, GA, 30309, US

(21) Appl. No.: 10/839,269

(22) Filed: May 5, 2004

(51) Int. Cl. A61B 17/56

(52) U.S. Cl. 606/72

(57) ABSTRACT

A soft tissue fixation kit comprises a flexible implant and an insertion tool for the implant. The implant is formed of a flexible biocompatible material and a rigid bone biocompatible fixation device. The insertion tool carries the implant percutaneously for insertion into selected soft tissue and bone. When the insertion tool is removed the implant is left in the selected tissue and anchored to the bone.

### TECHNICAL FIELD

This invention relates generally to soft tissue fixation to bone, and more particularly to corrections of soft tissue facial ligamentous relaxation by the insertion of biocompatible flexible implants having a cavity into which fibrous tissue can grow and thus which predictably and stably augment tissue defects.

### BACKGROUND OF THE INVENTION

Current common face lift techniques involve elevation and tightening of the skin, and tightening of the dense connective tissue of the subcutaneous myo-aponeurotic system, which overlies the jaw anterior to the ear. This technique is effective for dealing with the laxity of skin and soft tissue along the jaw and neck. However it is not effective for laxity of tissue with the nose meets the region around the mouth. The fold created by laxity of the tissue of the cheek in this region is called the nasolabial fold. Although some surgeons have developed techniques involving complicated dissection in this region, the risks include damage to the nerve which closes the eyelids and to the nerve which provides sensation to face.

Although approximately 50 to 60,000 face lifts are performed annually (based on data from a 1996 survey), the vast majority of these procedures do not address this region of the face due to the complexity of dissection in this region or unacceptable scarring The device described in this paper helps to alleviate this problem and may help the standardized technique of percutaneous repair of nasolabial folds.

The properties of various synthetic implant materials have been reported when used in facial augmentation procedures, particularly for reconstructions. Synthetic implants have been used in augmentation procedures. The materials used include solid, medical-grade silicone rubber (“Silastic,” available from Dow-Corning Corp., Midland, Mich.), braided, multifilament PET (“Mersilene,” available from Ethicon Corp., Summerville, N.J.), polyamide mesh (“Supramid,” available from S. Jackson, Inc., Alexandria, Va.), polytetrafluoroethylene resin (“Teflon,” available from C. R. Bard, Inc., Billerica, Mass.), polytetrafluoroethylene carbon (“Proplast,” available from Vitek, Inc., Houston, Tex.), hydroxyapatite (available from Integrated Orbital Implants, San Diego, Calif.), and expanded, fibrillated polytetrafluoroethylene, or PTFE (“GoreTex,” available from W. L. Gore, Phoenix, Ariz.).

Maas et al. compared the gross behavior of various currently used implant materials for facial bone augmentation at different sites in dogs. The authors concluded that the site of implantation and implant movement were important factors in determining the nature of the tissue response and the fate of implants. Maas et al., “Comparison of Biomaterials for Facial Bone Augmentation,” Arch. Otolaryngol. Head Neck Surg., 116, pp. 551-556 (1990).

### SUMMARY OF THE INVENTION

In one aspect of the present invention, a soft tissue fixation kit comprises a flexible implant and an insertion tool for the implant. The cavity can permit the implant to be mounted or carried by the insertion tool, preferably so that the implant can be inserted and positioned subcutaneously into bony and soft tissue through a very small incision. The cavity can also serve for anchoring the implant by fibrous tissue ingrowth.

The implant is formed of a biocompatible material, and preferably has a cross-section between exterior and interior surfaces that is permeable to red blood cells. Preferred permeability is where the cross section has pores of between about 10 to about 50 mμ.

Additional objects, advantages, and novel features of the invention will be set forth in the description which follows and will also become apparent to those skilled in the art.

### BRIEF DESCRIPTION OF THE DRAWINGS

FIG. 1 is a front view of an implant for soft tissue fixation according to the present invention.

FIG. 2 is an isometric view of a device for inserting the implant of FIG. 1.

FIG. 3 is a front view of a portion of a human face, showing relevant facial anatomy.

FIG. 4 is a front view of the portion of the human face showing a first step in the procedure for inserting the implant of FIG. 1.

FIG. 5 is a cutaway view of the human face between the eye socket and the nasolabial folds illustrating a second step in the procedure for inserting the implant of FIG. 1.

FIG. 6 is a cutaway view of the human face between the eye socket and the nasolabial folds illustrating a third step in the procedure for inserting the implant of FIG. 1.

FIG. 7 is a cutaway view of the human face between the eye socket and the nasolabial folds illustrating a fourth step in the procedure for inserting the implant of FIG. 1.

FIG. 8 is a front view of the portion of the human face showing a fifth step in the procedure for inserting the implant of FIG. 1.

FIG. 9 is a cutaway view of the human face between the eye socket and the nasolabial folds illustrating a sixth step in the procedure for inserting the implant of FIG. 1.

### DETAILED DESCRIPTION OF THE DISCLOSED EMBODIMENT

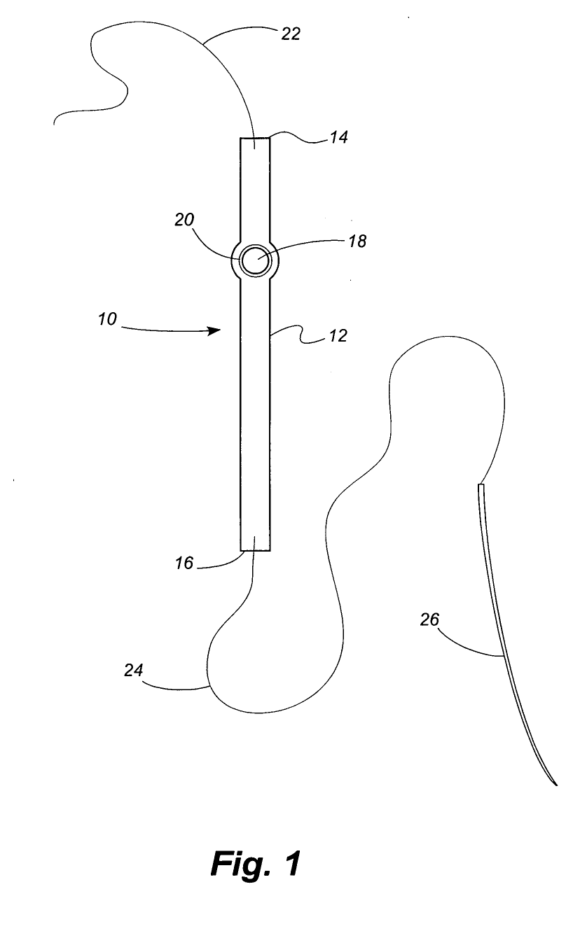

Referring now to the drawings, in which like numerals indicate like elements throughout the several views, FIG. 1 shows an implant 10 for use in affixing soft tissue to bone. The implant 10 has a body portion 12 with an upper end 14 and a lower end 16. An aperture 18 is formed in an upper middle section of the implant body 12. Preferably a grommet 20 is provided to reinforce the aperture 18.

The implant body 12 is formed of a biocompatible material, and preferably has a cross-section between exterior and interior surfaces that is permeable to red blood cells. Preferred permeability is where the cross section has pores of between about 10 to about 50 ml. The implant body of the disclosed embodiment is formed from expanded, fibrillated polytetrafluoroethylene, or PTFE (“GoreTex,” available from W. L. Gore, Phoenix, Ariz.).

An upper suture 22 is attached at one end to the upper end 14 of the implant body. A lower suture 24 is similarly attached at one end to the lower end 16 of the implant body. A curved surgical needle 26, such as a Keith needle, is attached to the opposite end of the lower suture 24.

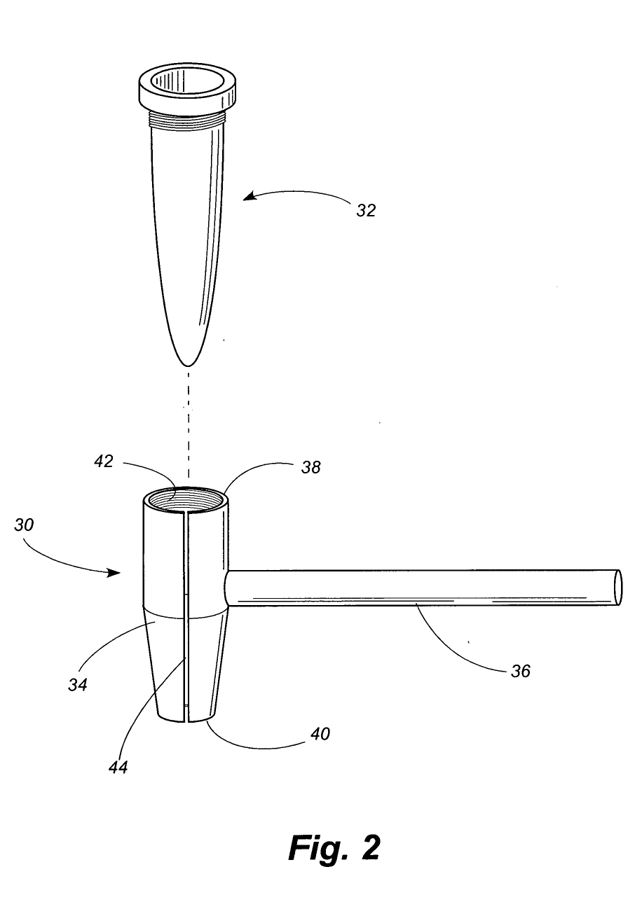

Referring now to FIG. 2, a device for installing the implant 10 in the tissues of a patient comprises a sleeve 30 and a cooperating trocar 32. The sleeve 30 comprises a body portion 34 and a laterally extending handle 36 to facilitate a physician holding the sleeve in a predetermined position. The sleeve 30 is tapered and has an upper end 38 and a lower end 40 that is smaller than the upper end. The interior surface of the sleeve body 34 has threads 42 formed adjacent the upper end 38.

The trocar 32 is configured to be received within the sleeve 30 so as to occlude the lower end 40 to prevent tissue from entering. Threads 44 adjacent the upper end of the trocar engage corresponding threads 42 on the inner surface of the sleeve to couple the trocar and sleeve together in cooperative relation.

Applied Anatomy:

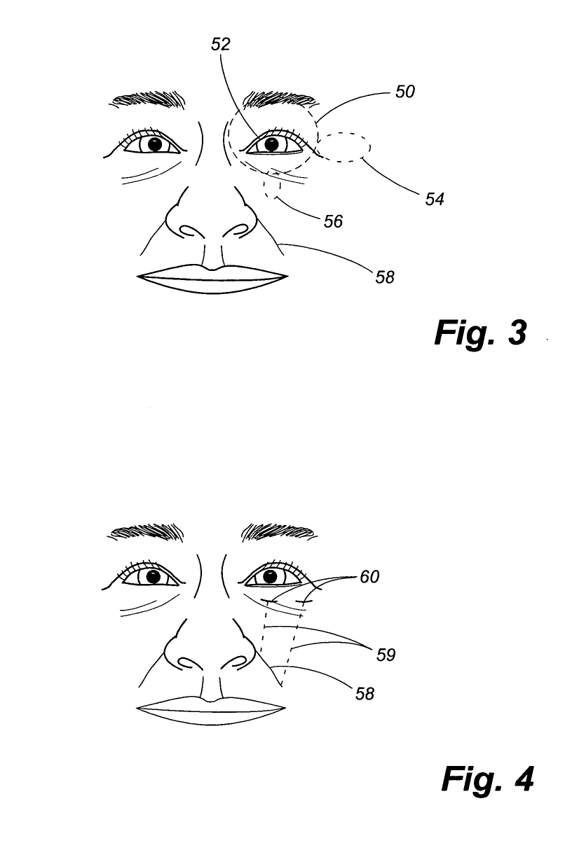

Referring now to FIG. 3, the orbital rim 50 is the bony structure surrounding the eye 52 which provides it supports and protection. The muscles which closes the eye are innervated by the facial nerve. This nerve has multiple branches which supply the eye (the most important contribution to protect in the treatment of the midface). It enters the muscles in the eye at a location 54 lateral to the orbital rim 50. Inferior to the eye, the infraorbital nerve provides sensation to the eye and exits the orbit through the orbital rim 50 at approximately the region 56 below the pupil of the eye gazing straight ahead. The nasolabial fold is shown at 58.

Technique of Insertion:

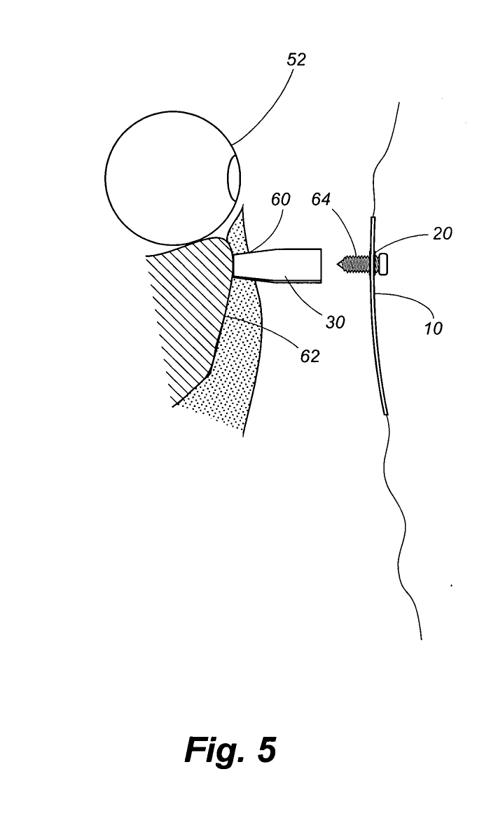

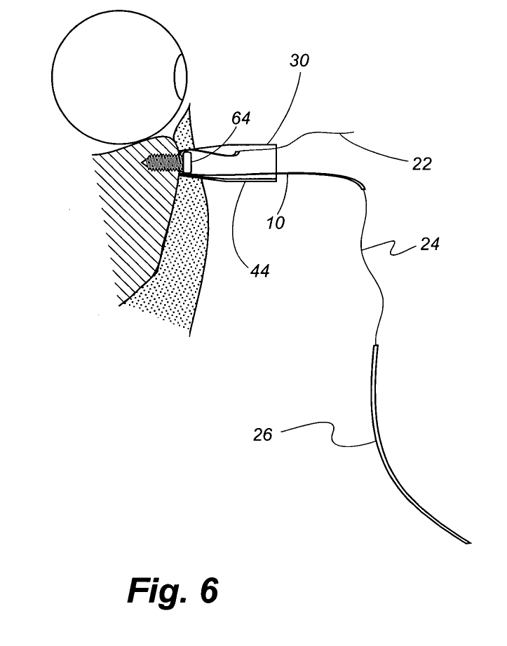

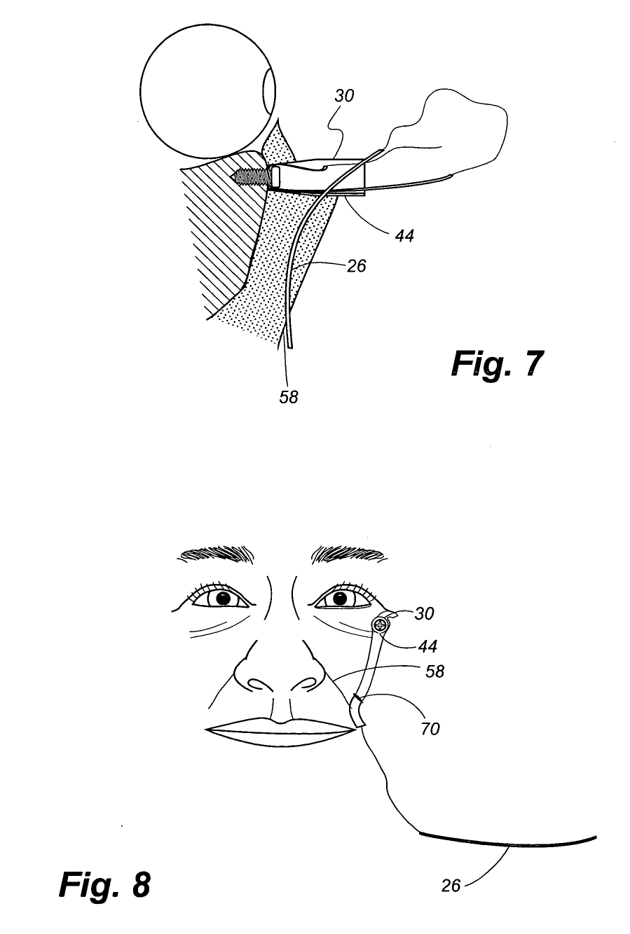

The technique of inserting the implant 10 will now be explained with reference to FIGS. 4-9. After first locating the surface anatomy as explained above in reference to FIG. 3, the surgeon will determine region of the above described nerves and the bony orbital rim. The desired vectors 59 of cheek elevation are determined, and location of 25 mm stab incisions 60 at the inferior aspect of the orbital rim will be ascertained as shown in FIG. 4. After injection of local anesthesia with epinephrine, a skin incision 60 is made, and the trocar 32, inserted into its sleeve 30, is used to dissect to the orbital rim. The trocar 32 is then removed from the sleeve 30, while holding the trocar sleeve firm to the bone 62, as shown in FIG. 5. A hand or powered drill is then used, and the implant 10, with a 1.7 mm screw 64 through the center of the grommet 20 in the implant, is anchored to the bone 62, as shown in FIG. 6. The Keith needle 26 is then inserted into the sleeve 30 and passed through the slot 44 in the sleeve in a plane in the soft tissues just superficial to the bone or just subdermal to exit just above the nasolabial fold 58, as illustrated in FIG. 7.

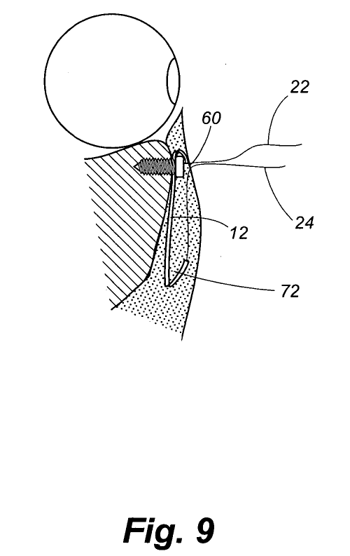

With reference now to FIG. 8, the needle 26 is then reversed and passed back through its exit hole 70 above the nasolabial fold 58, and back into the groove 44 in the trocar sleeve 30. The trocar sleeve 30 is then removed, and the sutures 22, 24 tied sufficiently tightly to elevate the cheek fat pad 72, as shown in FIG. 9. The skin incision 60 is then closed.

The foregoing procedure is then repeated at the location of the other stab incision 60.

Unless otherwise stated, terms used herein such as “top,” “bottom,” “upper,” “lower,” “left,” “right,” “front,” “back,” “proximal,” “distal,” and the like are used only for convenience of description and are not intended to limit the invention to any particular orientation.

Finally, it will be understood that the preferred embodiment has been disclosed by way of example, and that other modifications may occur to those skilled in the art without departing from the scope and spirit of the appended claims.

1. A device for implanting within the tissues of a patient, comprising:
a body having upper and lower ends;
an aperture formed in said body;
a first suture having a first end attached to said upper end of said body;
a second suture having a first end attached to said lower end of said body; and
a curved surgical needle attached to a second end of said second suture.

2. The device of claim 1, wherein said aperture has edges, and further comprising a grommet affixed to said body so as to reinforce said edges of said aperture.

3. The device of claim 1, wherein said body is permeable to red blood cells.

4. The device of claim 3, wherein a cross-section of said body has pores of between about 10 to about 50 ml.

5. The device of claim 3, wherein said body is comprised of expanded, fibrillated polytetrafluoroethylene.

6. The device of claim 3, wherein said body has a length of from 1.2 to 1.8 times the distance between a patient's bony orbital rim and said patient's nasolabial fold.

7. The device of claim 3, wherein said body has a length of approximately 1.5 times the distance between a patient's bony orbital rim and said patient's nasolabial fold.

8. A device for inserting an implant for affixing soft tissue to bone, comprising:
a tapered sleeve having a first end and having a second end smaller than said first end;
a trocar dimensioned to be received within said sleeve in cooperative relationship for closing said second end of said sleeve;
means for coupling said trocar and said sleeve in said cooperative relationship; and
a longitudinal slot formed in said tapered sleeve, said longitudinal slot being dimensioned to receive a surgical needle therethrough.

9. The device of claim 8, wherein said longitudinal slot extends from said first end of said sleeve to said second end of said sleeve.

10. The device of claim 8, further comprising a handle mounted to said sleeve to facilitate a physician holding said sleeve in a predetermined position.

11. The device of claim 8, wherein said means for coupling said trocar and said sleeve in said cooperative relationship comprises:
threads formed on said interior surface of said sleeve at said first end of said sleeve; and
cooperative threads formed on an exterior surface of said trocar at a first end of said trocar.

12. A method for affixing soft tissue to bone, comprising the steps of:
forming an incision at an anchor location on a patient;
inserting a forward end of a sleeve and trocar assembly into said incision;
using said forward end of said sleeve and trocar assembly, dissecting the tissues of said patient between said incision and an underlying location on a bone;
withdrawing said trocar from said sleeve;
inserting an implant into a rearward end of said sleeve;
inserting an anchor screw into said rearward end of said sleeve and through said implant;
anchoring said screw into said bone so as to attach said implant to said bone;
inserting a needle attached to a first end of said implant through a longitudinal slot in said sleeve to feed said first end of said implant into the tissues of said patient adjacent said sleeve; and
wrapping said first end of said implant around a portion of tissue to anchor said tissue to said bone.
